(19) United States
(12) Patent Application Publication
(10) Pub. No.: US 2023/0145646 A1
(43) Pub. Date: May 11, 2023

(54) METHOD OF MANUFACTURING LATEX RUBBER ARTICLES

(71) Applicant: Best Perwira Gloves Sdn Bhd, Nilai (MY)

(72) Inventors: Krzysztof KOZIOL, Cranfield (GB); Eva PELÁEZ-ÁLVAREZ, Cranfield (GB)

(21) Appl. No.: 17/916,177

(22) PCT Filed: Mar. 30, 2021
(86) PCT No.: PCT/IB2021/052614

§ 371 Date: Sep. 30, 2022

(30) Foreign Application Priority Data
Mar. 30, 2020 (GB) 2004571.2

(51) Int. Cl. C08J 5/02; C08K 5/16; A61B 42/10; A61L 31/04; B29C 41/08; B29C 41/22; A41D 19/00; B29C 35/02

(52) U.S. Cl. CPC C08J 5/02; C08K 5/16; A61B 42/10; A61L 31/049; B29C 41/085; B29C 41/22; A41D 19/0058; B29C 35/02; C08J 2321/02; B29K 2105/0064

(57) ABSTRACT

A method of manufacturing a latex rubber article comprises providing a former wherein at least a part of the former comprises a mould surface that forms the shape of the latex rubber article, and applying liquid latex to the mould surface using an applicator that is configured to apply the liquid latex to an applicator area that is smaller than the mould surface. The method further comprises providing relative movement between the applicator and the former to produce a latex coating that covers the mould surface, curing the latex coating on the former to form the latex rubber article, and removing the latex rubber article from the former.

The present invention relates to a method of manufacturing latex rubber articles. In particular, but not exclusively, the invention relates to a method of manufacturing latex rubber gloves, including natural rubber latex gloves. The invention also relates to latex rubber articles manufactured by the method, including latex rubber gloves and other articles.

Latex is a stable dispersion of polymer particles in an aqueous medium. Natural latex containing polymers of isoprene may be extracted from trees including, in particular, the rubber tree (Hevea brasiliensis). Synthetic latexes are also available. Natural latex, once extracted from its source, is concentrated and mixed with other chemicals to prepare a colloidal suspension of polymer particles. Latex used for glove manufacturing typically contains approximately 60% weight solid polymer particles.

Latex rubber gloves have been mass produced for decades using a dipping process, in which hand-shaped moulds (called formers) are covered with a coagulant and then submerged in a bath containing the liquid latex. When dipped in the bath, the latex adheres to the former forming a layer. The formers are removed from the bath and the latex is cured in an oven. The moulded gloves formed by this process are removed from the formers and may undergo a number of post processing steps and sterilisation, to form finished products. Typically, the gloves are removed from the formers manually by an operator or automatically by a burst of pressured air provided through hollow formers to blow the gloves off the formers.

A problem with the traditional moulding process based on dipping is that it can be wasteful of raw materials. The liquid latex in the dipping bath tends to coagulate over time and may eventually become unusable. The changing consistency of the liquid latex can also lead to quality control issues.

The conventional manufacturing process also does not allow for customisation of the moulded articles. For example, in the case of surgical gloves, it may be desirable to have a reduced thickness in the finger regions for increased sensitivity and dexterity and an increased thickness in the cuff region or the dorsal region of the glove (i.e. the back of the hand) for increased strength. However, the traditional dipping process does not allow the thickness of the rubber to be controlled in this way. In fact, a common problem with surgical gloves made by the dipping process is that the rubber tends to be thicker in the fingertip region and thinner in the cuff region, owing to the fact that when the formers are removed from the dipping bath the liquid rubber tends to run downwards towards the fingertips.

It may also be desirable to vary other characteristics in different regions of the glove, such as puncture or stab resistance, but again this is not possible with traditional manufacturing methods.

A problem with traditional surgical gloves is that they provide virtually no protection against ionising radiation, for example X-rays, which may be used during surgery for positioning pins in bones or visualising blood vessels. Although shielding aprons may be provided to protect the bodies of the surgical team, their hands remain unprotected and can receive potentially harmful levels of radiation exposure. Attempts have been made to incorporate shielding substances such as bismuth oxide (Bi₂O₃), by adding the shielding substance to the liquid latex in the dipping bath, but this has not met with success as the substances tend to separate in the dipping bath, leading to uneven and unpredictable distribution in the finished product. It can also lead to increased wastage of the raw latex material.

Similarly, attempts have been made to increase the electrical conductivity of latex rubber gloves to prevent a build-up of static electricity, by incorporating an electrically conductive material such as carbon powder in the liquid latex. However, these attempts have run into similar problems with separation of the materials in the dipping bath, leading to uneven distribution in the finished product and increased wastage of the raw material.

A need exists therefore for a method of manufacturing latex rubber gloves and other latex rubber articles that addresses one or more of the aforesaid problems and/or other problems associated with existing manufacturing methods, and to provide latex rubber articles, such as latex rubber gloves, having improved properties or that can be customised according to particular requirements.

CN108783675 discloses a knitting glove and a method of manufacturing thereof. The knitting glove is made by spraying a glove film-forming material comprising butadiene rubber, latex, polyamide fiber, medical stone fiber, graphene fiber, polyvinyl alcohol, and modified styrene-acrylic emulsion on a hand mold to form a 1.2-1.5-mm-thick film that is then dried in an environment with a temperature of 25-30° C. and an ambient humidity of 80-85% to form a preliminary glove. A 0.03-0.05-mm-thick protective layer comprising modified styrene-acrylic emulsion is sprayed onto the surface of the preliminary glove and drying is repeated at a temperature of 21-25° C. and ambient humidity of 88-90%. WO98/25747 discloses a method of manufacturing a thin-walled article by electrostatically spraying charged particles of an elastomeric composition into a chamber containing a rigid former having a conductive surface whereby the charged particles of the composition are attracted to the conductive surface of the former to form a coating on the former and subsequently consolidating the coating to produce the thin-walled article. A conveyer is used to convey formers into a chamber and charged particles are then sprayed into the chamber and are attracted to the former. The charged particles form a coating on the former, and after the former has been coated a heating means is activated to heat the former thereby drying the composition on the former and resulting in the thin-walled article.

WO91/03955 discloses a disposable protective glove for the human hand formed by providing a thin coating of liquid protective material on the surface of the hand, and converting the coating into a flexible, continuous film of protective material intimately contacting the surface of the hand by curing the coating on the hand.

According to one aspect of the present invention there is provided a method of manufacturing a latex rubber article, the method comprising:
a. providing a former wherein at least a part of the former comprises a mould surface that forms the shape of the latex rubber article;
b. applying liquid latex to the mould surface using an applicator, wherein the applicator is configured to apply the liquid latex to an applicator area that is smaller than the mould surface;
c. providing relative movement between the applicator and the former to produce a latex coating that covers the mould surface;
d. curing the latex coating on the former to form the latex rubber article or at least a first layer of the latex rubber article and repeating steps a-d to form the latex rubber article, and
e. removing the latex rubber article from the former.

The invention can reduce wastage of raw materials, as the liquid latex is applied directly to the former without leaving quantities of latex in a dipping tank. Changes in the consistency of the liquid latex can also be avoided, improving quality control.

The manufacturing process also allows for customisation of the moulded articles. For example, in the case of surgical gloves, it may be possible to provide a reduced thickness in the finger regions for increased sensitivity and dexterity and an increased thickness in the cuff region or the dorsal region of the glove for increased strength. The uniformity and predictability of thickness (where desired) can also be improved.

It may also be possible to vary other characteristics in different regions of the glove, such as puncture or stab resistance.

Advantageously, the mould surface comprises a three dimensional surface, to form a three dimensional article. The former may optionally have a longitudinal axis and the three dimensional mould surface may extend around the longitudinal axis to form an article that is at least partially cylindrical.

The manufacturing method may be automated or manually controlled. For example, the relative movement between the applicator and the former may be provided by a drive means that is automatically or manually controlled. For example, the relative movement between the applicator and the former may be automatically controlled via a controller, for example a computer or other electronic control device. Alternatively, the relative movement can be controlled by a human operator.

In an embodiment, the liquid latex comprises an aqueous dispersion that includes polymer particles in an amount ranging between about 40% wt. to about 70% wt., or about 45% wt. to about 65% wt., or about 50% wt. to 60% wt., relative to the total % wt. of the aqueous dispersion.

In an embodiment, the liquid latex comprises ammonia in an amount ranging from about 0.2% wt. to about 10% wt., or about 0.2% wt. to about 2% wt., or about 0.2% wt. to about 1% wt., relative to the total % wt. of the liquid latex solution.

The liquid latex does not necessarily have to consist of pure latex. For example, the liquid latex may include a blend of latex with other materials such as other low- or high-molecular weight polymers or oligomers.

In an embodiment, the liquid latex comprises an additive selected from the group comprising ceramic powders, carbon materials, nanomaterials, 2D materials, boron nitride, graphene, 1D materials, carbon nanotubes, bismuth oxide, iron oxide, ferrite and carbon. These functional additives may be selected to enhance certain properties or characteristics of the latex moulded article, for example to increase its strength or electrical conductivity, or to provide shielding against ionising radiation. Other functional additives may also be included, in addition to or as alternatives to the aforesaid components.

In an embodiment, the applicator area is less than 20 cm², or less than 10 cm², or less than 5 cm². The applicator area is the size of the area on the mould surface to which the applicator applies the liquid latex when there is no relative movement between the applicator and the mould surface. Where the applicator is a spray nozzle, the applicator area is the size of the spray cone at the point where the spray impacts the mould surface.

In an embodiment, the method further comprises applying the liquid latex to a plurality of application areas, the plurality of application areas comprising at least a first application area and a second application area.

In an embodiment, the method further comprises applying the liquid latex to the plurality of application areas simultaneously.

In an embodiment, the method further comprises applying the liquid latex to the first application area and the second application area to provide the first application area with a first portion of the latex coating and the second application area with a second portion of the latex coating.

In an embodiment, the first portion and the second portion comprise different thicknesses of coating. By providing different thicknesses of coating in different portions of the mould surface it is possible to manufacture a latex rubber glove for example with different thicknesses of latex in different regions of the glove. For example, in the case of a surgical glove or a medical examination glove, a reduced thickness can be provided in the fingertip regions for increased sensitive and an increased thickness can be provided in the cuff region for increased strength to reduce the risk of tearing when putting the glove on. Thus, the method provides a distinct advantage over traditional dipped gloves, which normally have a substantially uniform thickness and cannot be customised to vary the thickness in a controlled manner in specific regions of the glove.

In an embodiment, the method further comprises the first portion and the second portion comprising different liquid latex compositions, thereby providing different properties in different portions of the latex rubber article.

In an embodiment, the method further comprises overlapping the first application area at least partially with the second application area, to form a continuous layer.

In an embodiment, the method further comprises applying the liquid latex to the mould surface to provide a coating thickness of between 80 μm and 500 μm, preferably between 100 μm and 500 μm, more preferably between 150 μm and 250 μm.

Advantageously, the manufacturing process allows for the formation of a continuous layer with a coating thickness that is optimised for a given application so as to enable efficient use of material, achieve desired mechanical properties, and facilitate ease of removal of the latex rubber article from the former to form a free standing object.

In an embodiment, the method further comprises applying the liquid latex to the mould surface in a plurality of layers to produce the latex coating, the plurality of layers comprising at least a first layer and a second layer. In an embodiment, each layer has a thickness of between 10 μm and 200 μm, or between 20 μm and 150 μm, or between 40 μm and 100 μm.

In an embodiment, the method further comprises applying the liquid latex to the mould surface to form the first layer, and then applying the liquid latex to the first layer to form the second layer, such that layer-by-layer deposition is used to produce the latex coating.

Advantageously, the method comprising layer-by-layer deposition allows for a coating thickness that is not limited to a maximum thickness of an individual layer as the thickness of the coating can be increased by depositing further layers depending on the build cycle and as required by the application. In an embodiment, the coating thickness is between 80 μm and 500 μm, preferably between 100 μm and 500 μm, more preferably between 150 μm and 250 μm.

In an embodiment, the method further comprises applying the liquid latex to the first layer to form the second layer after the first layer has at least partially cured.

In an embodiment, the method further comprises applying a first liquid latex to form the first layer and applying a second liquid latex to form the second layer, the first liquid latex and the second liquid latex comprising different liquid latex compositions. Alternatively, two layers of different liquid latex mixtures can be co-deposited simultaneously onto the former. Optionally, the second layer comprises an additive selected from the group comprising ceramic powders, carbon materials, nanomaterials, 2D materials, boron nitride, graphene, 1D materials, carbon nanotubes, bismuth oxide, iron oxide, ferrite and carbon. These functional additives may be selected to enhance certain properties or characteristics of the latex moulded article, for example to increase its strength or electrical conductivity, or to provide shielding against ionising radiation. Other functional additives may also be included, in addition to or as alternatives to the aforesaid components. Optionally, at least one additional layer may be applied, the at least one additional layer comprising a liquid latex composition and/or one or more functional additives.

In an embodiment, the first layer and the second layer comprise different thicknesses.

In an embodiment, the former comprises a ceramic material.

In an embodiment, the former comprises a hand-shaped mould surface and the latex rubber article comprises a latex glove.

In an embodiment, the applicator comprises a spraying nozzle or a plurality of spraying nozzles. The liquid latex may thus be deposited as a spray, which may comprise nano-, micro- or milli-sized droplets, or combinations thereof. The droplets may for example be generated using a high velocity gas flow (for example, using an air brush), or by pressure or sonication or an electric field or by combinations of these and other methods. Alternatively, the applicator may comprise another kind of applicator device, such a pen-like contact applicator.

In an embodiment, the method further comprises adjusting the applicator relative to the former to alter the angle of application of the liquid latex to the mould surface.

In an embodiment, the method further comprises providing a plurality of applicators, wherein each applicator is independently adjustable relative to the former.

In an embodiment, the method further comprises heating the former to cure the liquid latex applied to the mould surface and form a layer of cured latex on the mould surface.

In an embodiment, the method further comprises applying additional liquid latex to the layer of cured latex on the mould surface.

In an embodiment, the method further comprises heating the former using an internal heater, for example an internal resistance heater.

In an embodiment, the method further comprises heating the former using an external heater, for example a heater that is external to the former.

In an embodiment, the method further comprises heating the former to a temperature in the range between 20° C. and 160° C., or between 20° C. and 100° C., or between 20° C. and 60° C.

In an embodiment, heating the former may include heating the former before and/or while applying liquid latex to the mould surface.

In an embodiment, the method further comprises heating the former to a first temperature while applying liquid latex to the former and heating the former to a second temperature after applying liquid latex to the former, wherein the second temperature is higher than the first temperature.

In an embodiment, the first temperature comprises heating the former using an internal heater or an external heater, and the second temperature comprises heating the former using the internal heater and an external heater.

In an embodiment, providing relative movement between the applicator and the former comprises moving the former, or moving the applicator, or moving both the former and the applicator.

In an embodiment, providing relative movement between the applicator and the former comprises providing relative rotation about an axis, for example a longitudinal axis of the former.

In an embodiment, providing relative movement between the former and the applicator further comprises providing relative movement in a direction that is substantially parallel or perpendicular to the axis.

According to another aspect of the invention there is provided a latex rubber article manufactured by a method as defined by any one of the preceding statements of invention.

In an embodiment, the latex rubber article is a latex rubber glove that comprises a plurality of glove portions, including a palmar portion, dorsal portion and a finger portion.

In an embodiment, at least one of said glove portions has a uniform thickness distribution with a standard deviation of less than 0.035, or less than 0.03, or less than 0.025.

Advantageously, the method allows for the formation of a latex rubber article for example a latex rubber glove with a uniform thickness distribution that is at least similar to that obtainable via conventional processes. The method comprising layer-by-layer deposition allows for a uniform thickness distribution that is at least similar to that obtainable via conventional processes. Therefore, the method allows for the formation of a latex rubber article for example a latex rubber glove comprising a one or more layers in which the uniformity in thickness of the layers is at least similar to that obtainable via conventional processes or other processes as will be appreciated by the person skilled in the art.

In an embodiment, the latex rubber glove comprises at least a first region and a second region, the first region and the second region comprising different thicknesses.

In an embodiment, the first region comprises the finger portion of the latex rubber glove, and the thickness is less in the first region than the second region.

In an embodiment, the second region comprises a cuff region of the latex rubber glove, and the thickness is greater in the second region than the first region.

In an embodiment, the latex rubber in at least one of the glove portions comprises an additive from a group comprising ceramic powders, carbon materials, nanomaterials, 2D materials, boron nitride, graphene, 1D materials, carbon nanotubes, bismuth oxide, iron oxide, ferrite and carbon.

According to another aspect of the invention, there is provided an apparatus for manufacturing the latex rubber article according to the method of claims 1 to 33 comprising a former wherein at least a part of the former comprises a mould surface that forms the shape of the latex rubber article, an applicator configured to apply liquid latex to an applicator area that is smaller than the mould surface, a drive means configured to provide relative movement between the applicator and the former to produce a latex coating that covers the mould surface, and a heater for heating the former configured to cure the latex coating on the former to form the latex rubber article or a first layer of the latex rubber article.

In an embodiment, the apparatus further comprises a chamber configured to house at least a part of the former and/or the applicator and/or the drive means and/or the heater.

In an embodiment, the heater comprises an internal heater, optionally an internal resistance heater.

In an embodiment, the heater comprises an external heater.

In an embodiment, the drive means comprises a controller configured to automatically control the relative movement between the applicator and the former.

In an embodiment, the apparatus further comprises a removal means configured to remove the latex rubber article from the former.

In an embodiment, the removal means comprises a pressured air supply configured to force the latex rubber article off the former. In an embodiment, a pressurised air supply is used to separate at least a part of the latex rubber article from the former in order to facilitate the removal of the latex rubber article from the former. In an embodiment, the removal means may comprise one or more metal plates located between the latex rubber article and the former to force the latex rubber article off the former.

Certain embodiments of the invention will now be described, by way of example, with reference to the accompanying drawings, wherein:

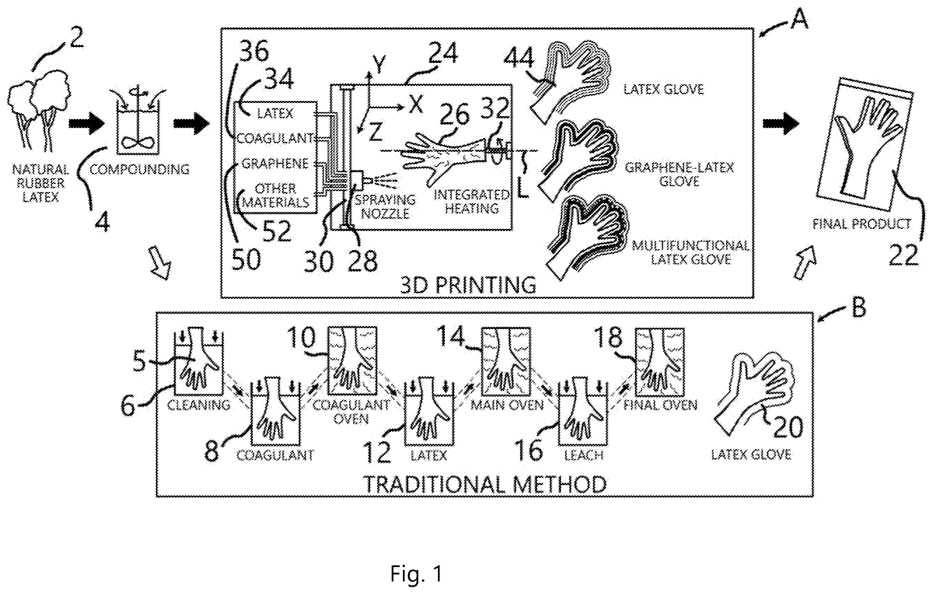

FIG. 1 illustrates schematically a manufacturing method according to an embodiment of the invention and, for comparison, the main steps of a conventional manufacturing method;

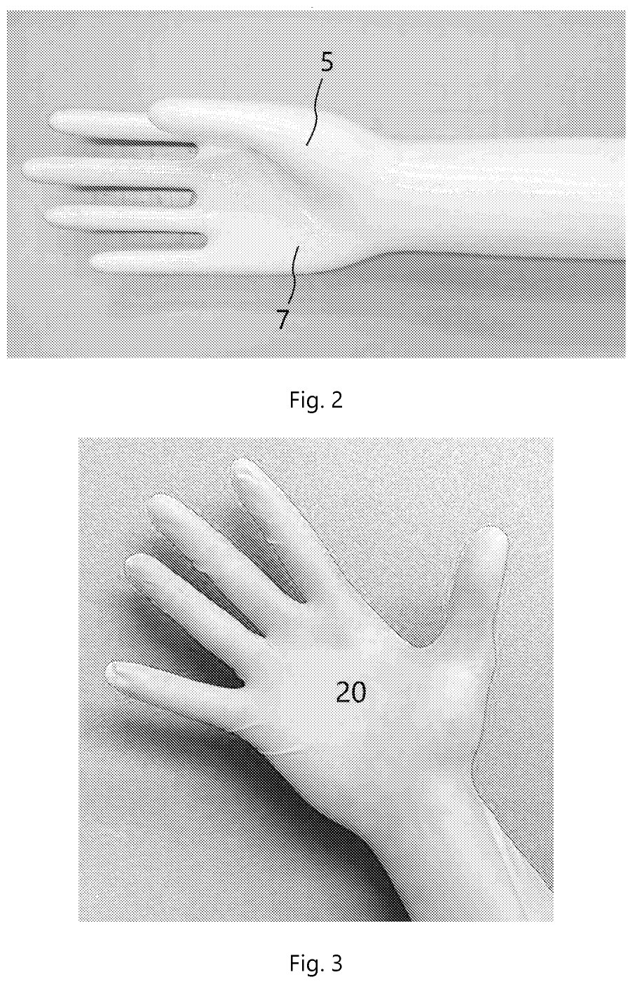

FIG. 2 is an illustration of a hand-shaped former used in a method of manufacturing a latex rubber glove;

FIG. 3 is an illustration of a latex rubber glove made using the method of manufacturing;

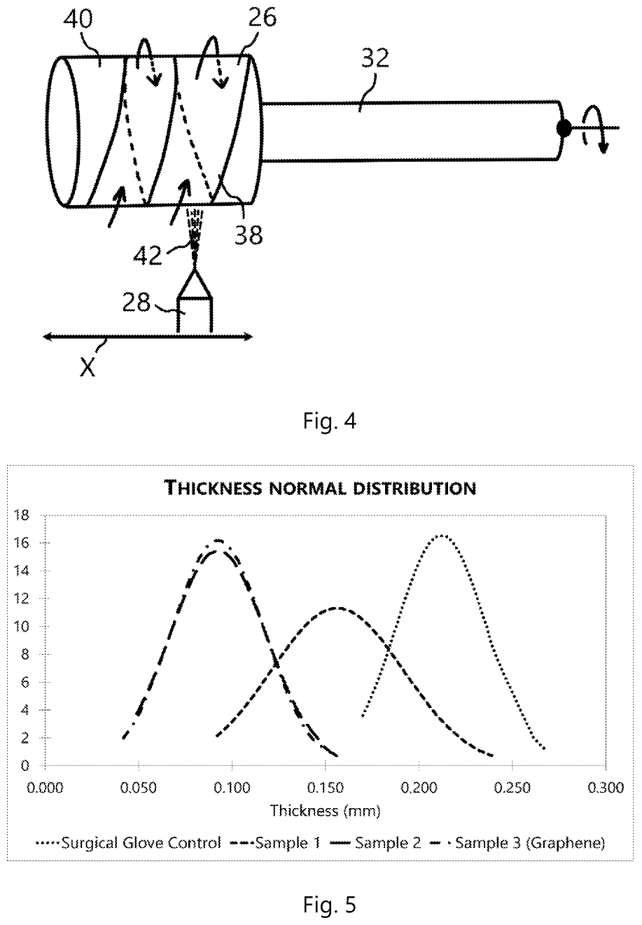

FIG. 4 illustrates schematically a 3D printing step comprising part of an embodiment of the invention;

FIG. 5 comprises a graph illustrating thickness measurements normal distributions for samples of moulded articles made by the manufacturing method;

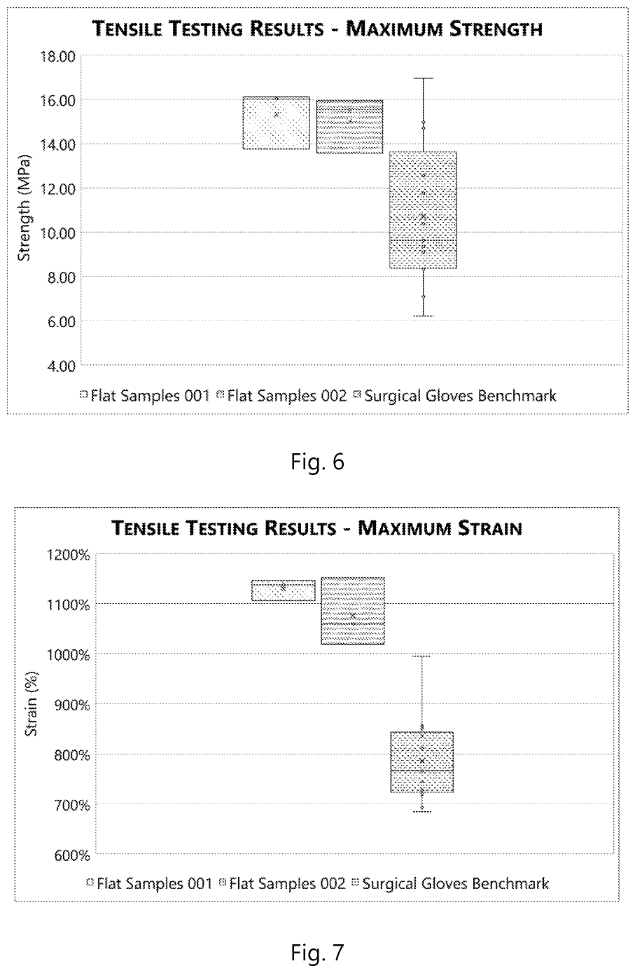

FIG. 6 illustrates graphically tensile testing results for maximum strength;

FIG. 7 illustrates graphically tensile testing results for maximum strain, and

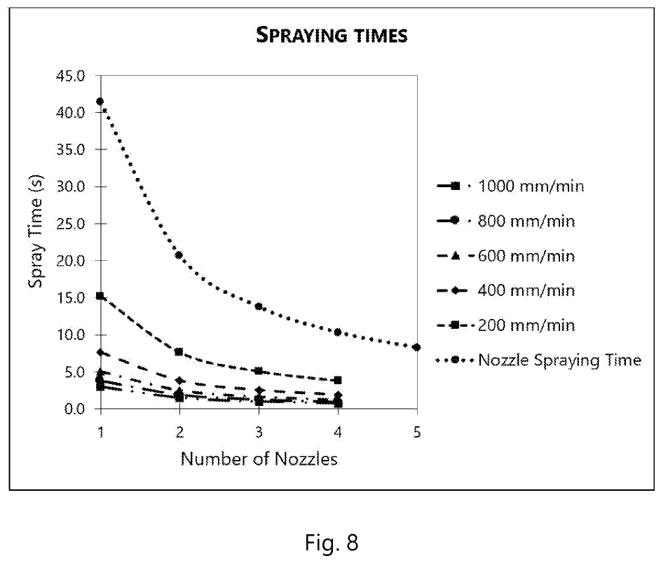

FIG. 8 illustrates graphically estimated spraying times for manufacturing methods using different numbers of spraying nozzles.

FIG. 1 illustrates schematically a manufacturing method according to an embodiment of the invention (A) and, for comparison, the main steps of a conventional manufacturing method (B). The manufacturing methods illustrated in FIG. 1 are used for the manufacture of latex rubber articles, which in these examples are latex rubber gloves. The method is also applicable to the manufacture of other latex rubber articles.

In the conventional manufacturing method (B), natural latex rubber is collected from rubber trees 2 and then concentrated by centrifugation and mixed with a small amount of ammonia (typically 0.5-1.0% wt) to help prevent premature coagulation. The concentrated liquid latex, typically containing about 60% wt solid matter, is mixed with other chemicals (e.g. stabilisers, vulcanising agents, curing agents and antioxidants) in a compounding device 4 to form a liquid latex mixture that will be used for manufacturing the gloves. This process is called “compounding”.

The gloves are moulded using hand-shaped ceramic moulds 5 (called “formers”), an example of which is shown in FIG. 2. The former has a 3 dimensional mould surface 7 to which the latex rubber is applied to form the glove. The former may be made for example of clay, which typically comprises silica, alumina or magnesia and sometimes appreciable quantities of potassium, sodium, and calcium.

The formers 5 are cleaned in a cleaning bath 6 and a coagulant is applied in a coagulant bath 8. The formers 5 are then dried in a coagulant oven 10. The formers 5 are then dipped into a latex bath 12, so that the mould surfaces of the formers are coated with the liquid latex mixture. After being removed from the latex bath 12, the formers 5 are placed in a gelling oven 14 that partially solidifies the latex. This is followed be a leaching process in which the formers 5 are dipped in a leaching bath 16 that removes chemicals and latex proteins, which are responsible for causing allergies. The formers 5 are then placed final oven 18, typically at a temperature in the range 100-120° C., to cure or vulcanise the latex rubber, which gives the gloves their final geometry and thickness. Various post processing steps can be applied, including dipping the gloves in a corn-starch solution to reduce tackiness (powdered common gloves) or a chlorination process plus coating to reduce protein content and tackiness, to form finished gloves 20. Finally, the gloves 20 are sterilised with gamma radiation or ethylene oxide, and wrapped in sterile packaging 22.

In a manufacturing method according to an embodiment of the invention (A), the latex rubber is collected from trees 2 and compounded in the conventional manner in a compounding device 4. The latex mixture may include additional water (typically about 20%) to make a thinner mixture that is more easily sprayed. The latex mixture is applied in an additive manufacturing machine 24 to a former 26 by a suitable additive manufacturing technique, for example by 3D printing or 3D spraying. The liquid latex mixture may include pre-vulcanised material formed by heating liquid latex compounding. This can lead to improved mechanical properties in the latex rubber product and may also allow the compound to remain stable for longer.

The term “additive manufacturing” as used herein is defined by the standard ISO/ASTM 52900:2015 as “the process of joining materials to make parts from 3D model data, usually layer upon layer, as opposed to subtractive manufacturing and formative manufacturing methodologies”. Accordingly, a manufacturing method according to the invention provides the advantages of additive manufacturing including the principles of single-step and multi-step processes within a process chamber of the additive manufacturing machine 24.

The term “3D printing” is used herein in a broad sense to include printing by spraying, wherein material is applied to a mould surface using an applicator (for example a spray nozzle) and the applicator is configured to apply the material to an applicator area that is smaller than the mould surface. Relative movement is provided between the applicator and the mould surface to produce a coating that covers the mould surface.

An embodiment of a manufacturing method according to the invention is illustrated in FIG. 1 as method (A). In this method, an applicator comprising a spraying nozzle 28 is mounted on a robotic support structure 30 that enables movement of the nozzle 28 relative to a former 26. The support structure 30 may optionally be configured to provide for movement relative to the former 26 in one or more of the following directions:
1. In a longitudinal direction X (for example, parallel to the longitudinal axis L of the former 26)
2. In a first transverse direction Y that is perpendicular to the longitudinal direction X (for example, a vertical direction)
3. In a second transverse direction Z that is perpendicular to the longitudinal direction X (for example, in a horizontal direction).

In addition, the nozzle 28 may optionally be configured to rotate about 1 or more of the axes X, Y, Z, to adjust the angle of the spray relative to the surface of the former.

Optionally, the former 26 having a mould surface may also be mounted on a support structure 32 that enables movement of the former 26 relative to the nozzle 28. For example, as illustrated in FIG. 1, the former 26 may be configured for rotation about the longitudinal axis L of the former 26.

The spraying nozzle 28 is configured to receive liquid latex from a liquid latex source 34, and a coagulant from a coagulant source 36. The liquid latex and the coagulant are mixed in the spraying nozzle 28 and applied as a spray to the mould surface of the former 26. A relatively narrow spray is produced by the nozzle 28 so that the mixture of liquid latex and coagulant is applied to an applicator area that is smaller than the mould surface of the former 26. For example, the applicator area may be less than 20 cm², preferably less than 10 cm² and more preferably less than 5 cm². Typically, the applicator area may be about 2 cm². For a rubber glove, the mould surface of the former 26 may typically be about 400 cm². Alternatively, the liquid latex and coagulant are not mixed in the nozzle but applied separately to the former 26. Preferably, the coagulant is first applied to the mould surface of the former 26 and the liquid latex is subsequently applied to the mould surface of the former 26. The spraying nozzle 28 is configured to first receive the coagulant from a coagulant source 36 to apply the coagulant to the mould surface of the former 26 and then liquid latex from a liquid latex source 34 to apply the liquid latex source 34 to the mould surface of the former 26. Alternatively, a plurality of spraying nozzles may be arranged such that a first spraying nozzle is configured to receive the coagulant from a coagulant source 36 to apply the coagulant to the mould surface of the former 26, and a second spraying nozzle is configured to receive the liquid latex from a liquid latex source 34 to apply the liquid latex source 34 to the mould surface of the former 26. While the mixture of liquid latex and/or coagulant is being applied to the mould surface of the former 26, relative movement is provided between the nozzle 28 and the former 26 so that the applicator area moves over the mould surface of the former 26. This relative movement can be provided by moving the nozzle 28, or by moving the former 26, or by moving both the nozzle 28 and the former 26. The relative movement is controlled, preferably by a control unit (for example a computer running a control program, e.g. a G-code) to ensure that the applicator area moves over the entire mould surface of the former 26, thereby building up a continuous layer of the latex/coagulant mixture that covers the mould surface. If required, a plurality of layers of the mixture may be applied, to build up a multi-layer moulded article.

Various different patterns of relative movement between the nozzle 28 and the former 26 can be provided to ensure that the latex/coagulant mixture that covers the mould surface. One example is illustrated in FIG. 2. In this example, the former 26 is rotated continuously around the longitudinal axis Y of the former while the nozzle 28 is moved longitudinally in the direction of axis X, parallel to the longitudinal axis Y. This produces a helical spray pattern 38 on the mould surface 40 of the former 26. By controlling the speeds of the rotational and longitudinal movements of the former 26 and the nozzle 28, the helical paths of the spray 42 over the mould surface 40 can be made to overlap, producing a continuous layer of latex that covers the mould surface 40. Numerous other patterns of relative movement between the nozzle 28 and the former 26 can also be provided to ensure that the latex/coagulant mixture that covers the mould surface 40.

It may also be possible to adjust the distance between the spray nozzle and the mould surface of the former, and/or the cone angle of the spray emerging from the spray nozzle to adjust the applicator area (the size of the spray when it reaches the mould surface of the former). For example, the distance from the spray nozzle to the mould surface of the former may typically be from 20 mm to 200 mm, preferably from 50 mm to 150 mm, and more preferably about 100 mm, producing an applicator area of less than 20 cm², preferably less than 10 cm², more preferably less than 5 cm².

It is also possible to use multiple applicators (e.g. multiple spraying nozzles) simultaneously, to speed up the manufacturing process. Alternative applicators may also be used, including for example pen-like applicator devices that apply liquid latex by contact with the mould surface of the former, or other known 3D printing techniques.

An embodiment of a manufacturing method according to the invention allows for the formation of a continuous latex layer with a uniform layer thickness or a thickness distribution that is determined by the relative movement between the applicator and the former. Preferably, automated control, for example, via a controller, for example a computer or other electronic control device allows for control of the thickness distribution.

The applicator may be used to deposit the liquid latex in a plurality of application areas on the mould surface to form a first application area with a first portion of the latex coating and the second application area with a second portion of the latex coating such that the first portion and the second portion comprise different thicknesses of coating. In addition, the first portion and the second portion may be deposited with different compositions, thereby providing different properties in different portions of the latex rubber article.

In another embodiment of a manufacturing method according to the invention, a continuous latex layer with a uniform layer thickness or a thickness distribution that is determined by the relative movement between the applicator and the former may be achieved by applying the liquid latex to the mould surface in a plurality of layers to produce the latex coating to form, for example, at least a first layer and a second layer. In this embodiment, the liquid latex is applied to the mould surface to form the first layer, and it is then applied over the first layer to form the second layer, such that layer-by-layer deposition is used to produce the latex coating. In addition, the first layer and the second layer may be deposited with different compositions, thereby providing different properties in different layers of the latex rubber article. The layer-by-layer deposition allows for the formation of different layers of different thicknesses depending on the application. For example, the first layer and the second layer may be deposited with different thicknesses and/or compositions.

Preferably, the application areas and/or the number of layers in a respective application area are controlled to form the continuous coating with uniform or varying distributions of both thickness and material properties.

The manufacturing method according to the invention may allow for the formation of a latex rubber article having different thicknesses in different portions of the latex rubber article. For example, the latex rubber article may have a uniform or non-uniform overall thickness distribution and may be made up of a plurality of layers in which each of the plurality of layers may have a uniform or non-uniform thickness. The application of liquid latex to the mould surface of the former 26 can provide a latex coating with one or more portions of uniform or non-uniform thicknesses depending on the application. The latex rubber article may be made up of for example a first portion and a second portion which have different thicknesses to each other. The latex rubber article can include one or more portions of different compositions. The method allows for the formation of a latex rubber article with a plurality of portions of different thicknesses and/or compositions as required by a given application.

The former 26 is preferably heated, which causes the liquid latex/coagulant mixture to cure or vulcanise on the mould surface of the former 26. Alternatively, if pre-vulcanised latex is used, the former 26 may be heated to dry the latex on the mould surface. Heating can be applied before and/or during and/or after applying the latex coagulant mixture to the mould surface. Heating can be provided for example by an internal heater, for example an electrical resistance heater, or by an external heater, for example an infrared lamp, or another external heating device. The former 26 can be hollow or can include a hollow section to accommodate the internal heater. Preferably, the former is pre-heated before the latex rubber is added and heat is continuously applied while the liquid latex is being applied, so that the latex starts to cure immediately as it contacts the mould surface of the former. This ensures that curing starts immediately, which reduces the risk of the liquid latex running over the mould surface and thereby affecting the thickness of the rubber. Where multiple layers of liquid latex are applied the heating also helps to ensure that each layer is at least partially cured before another layer is applied on top of that layer. Alternatively, multiple layers can be applied on top of one another without curing.

In an embodiment, the former is heated to a temperature in the range between 20° C. and 160° C., preferably between 20° C. and 100° C., or more preferably between 20° C. and 60° C. Heating the former to enable curing or drying on the former avoids the need for an external oven to complete curing/drying of the rubber, thereby speeding up the manufacturing process.

In an embodiment, the deposition of the liquid latex and/or coagulant mixture on the surface of the former and the heating of the former 26 to enable curing of the coating are processes that can be performed simultaneously. The step of curing the coating on the former 26 does not necessarily have to occur after the application of the mixture to the mould surface. Instead, the deposition and heating processes can occur simultaneously. This can allow for the rapid curing of the coating. The simultaneous deposition and heating processes can occur within the same process chamber alleviating the need to perform the processes in separate chambers.

During the deposition process, the heating can be provided for example by an internal heater integrated within the former 26, for example an electrical resistance heater, or by an external heater, for example an infrared lamp, or another external heating device. Alternatively, the heating can be applied before and/or during the application of the liquid latex and/or coagulant mixture to the mould surface of the former 26. The heating can commence prior to the deposition process to adequately pre-heat the former to a suitable temperature before the deposition process to ensure that when the deposition process commences the former 26 is at a suitable temperature enabling the rapid curing of the coating which occurs immediately upon deposition on the mould surface or another layer.

During the deposition process, the former 26 is heated to a first temperature in the range between 20° C. and 160° C., preferably between 20° C. and 100° C., or more preferably between 20° C. and 60° C. Upon completion of the deposition process, the former 26 is heated to a second temperature in the range between 20° C. and 160° C., preferably between 20° C. and 100° C. The second temperature is higher than the first temperature such that the former 26 is kept at a relatively lower temperature during the deposition and is then heated to a relatively higher temperature after the deposition is completed. In this way, rapid drying is achieved during the deposition process and post-deposition vulcanisation is promoted after the deposition process is completed, thereby reducing the overall time for the coating formation and the production of the latex rubber article.

In embodiments where multiple layers are applied, the former 26 is heated to a first temperature such that during the deposition of a subsequent layer, the former 26 is heated to a temperature in the range between 20° C. and 160° C., preferably between 20° C. and 100° C., or more preferably between 20° C. and 60° C. The simultaneous deposition and heating processes can enable the multiple layers to be deposited in a time-efficient manner as each layer is rapidly cured. The simultaneous heating of the former during deposition allows for the drying of the layer/coating while material is being applied/deposited on the former 26. Upon completion of the deposition process, the former 26 can then be heated to a second temperature in the range between 20° C. and 160° C., preferably between 20° C. and 100° C. The second temperature is higher than the first temperature such that the former 26 is kept at a relatively lower temperature during the deposition process and is then heated to a relatively higher temperature after the deposition process is completed. In this way, rapid drying is achieved during deposition, and post-deposition vulcanisation is promoted after the deposition is completed, thereby reducing the overall time taken for the coating formation and the production of the latex rubber article.

The liquid latex deposited on the mould surface of the former 26 may include pre-vulcanised latex material. Advantageously, this can provide an improvement in the overall mechanical properties of the latex rubber article. For example, the heating of the deposited liquid latex and/or coagulant mixture during or after the deposition process in which the liquid latex and/or coagulant mixture includes a pre-vulcanised material may provide improved mechanical properties. In specific embodiments, a liquid latex and/or coagulant mixture that includes pre-vulcanised material is applied to the former 26 and upon completion of the deposition process is subsequently heated. In other embodiments, a liquid latex and/or coagulant mixture that includes pre-vulcanised material is applied to the former 26 and is heated during the deposition process. The inclusion of a pre-vulcanised material in the liquid latex and/or coagulant mixture may improve the overall mechanical properties of the latex rubber article. Preferably, heating of the former 26 during the deposition process can be provided for example by the internal heater, for example an electrical resistance heater or by an external heater, for example an infrared lamp, or another external heating device. Preferably, heating of the former 26 after the deposition process can be provided for example by the internal heater, for example an electrical resistance heater, and by an external heater, for example an infrared lamp, or another external heating device. Alternative heating means as will be appreciated by those skilled in the art can include an ultra-violet (UV) lamp for curing UV curable materials. Preferably, a deposition temperature which may use the internal heater can be increased to a post-deposition vulcanisation temperature through the addition of the external heater.

A moulded latex rubber article, for example a glove 44, can thus be formed and cured/vulcanised/dried in a single continuous process. After forming, the article/glove 44 can be removed from the former 26 and optionally subjected to conventional post processing processes, sterilisation and packing to form the finished product 22.

Optionally, the applicator (e.g. spraying nozzle 28) may be configured to receive one or more functional additives that change or enhance certain physical properties of the moulded articles. These functional additives may comprise substances/materials that are mixed with the liquid latex and coagulant in the spraying nozzle 28 before being applied to the mould surface 40 of the former 26. Alternatively, the liquid latex can be co-sprayed onto the former with additives in the form of air-born powders.

For example, the spraying nozzle may be configured to receive graphene from a graphene source 50. The graphene can increase them mechanical strength of the latex rubber article and/or its electrical conductivity. This can reduce the risk of tearing and/or allow thinner layers to be used, allowing for increased sensitivity and/or dexterity. The increased electrical conductivity can provide protection against the build-up of static electricity.

The applicator may also or alternatively be configured to receive other substances or materials from another material source 52. These other substances/materials may include functional additives that change or enhance certain physical properties of the moulded articles and may include, for example, bismuth oxide for protection against ionising radiation, carbon powder for increased electrical conductivity, or other materials such as ceramic powder, nano materials, 2D materials such as boron nitride or 1D materials such as carbon nanotubes, or any other powders such as iron oxide, ferrite etc.

The functional additives may be applied uniformly over the whole of the mould surface of the former, or they may be applied selectively, or at different concentrations, in different regions of the former. For example, where bismuth oxide is applied for protection against ionising radiation, this may be applied preferentially or exclusively in regions that are exposed to higher levels of radiation—for example the dorsal region of the glove. A layer of bismuth oxide may be applied for example on the dorsal region of the glove as a layer built on top of a layer of natural rubber latex. This may be useful for surgeons, for example, to provide protection against radiation while leaving the distal region of the hand and fingertips relatively free so as to not limit dexterity of the wearer. Where graphene is added to increase the strength of the glove, it may be applied preferentially or exclusively in regions that require greater strength—for example in the cuff region, or in regions where strength is required without increasing the thickness of the glove—for example in the fingertip regions.

The functional additives may be applied in all layers of a multi-layer glove or in only one or more layers (the other layer or layers being constructed either from pure rubber latex or from rubber latex that includes one or more other additives). A layer containing a functional additive may cover the whole mould surface of the former or only part of the mould surface.

The manufacturing method according to the invention can allow for the formation of a latex rubber article for example a latex rubber glove that includes at least a first region and a second region in which the first and second regions have different thicknesses to each other. Different portions of a latex rubber glove can include for example a palmar portion, a dorsal portion and a finger portion. These portions of a latex rubber glove can be made of different thicknesses and/or compositions depending on requirements. For example, the first region and the second region can have different thicknesses and the thickness may be more or less in the first region as compared with that in the second region. The manufacturing method according to the invention allows for many possible combinations of regions and/or layers of the latex rubber article in which each of the regions and/or layers can have different thicknesses and/or compositions.

Glove samples made using the process described above have been tested for thickness and strength. The results of those tests are set out below.

### Thickness Measurement

Thickness measurements were performed on glove samples using a digital micrometer. Four samples were tested: a commercial glove made by a dipping process (Surgical Glove Control), and three 3D printed gloves made using first, second and third printing protocols (G-codes), which were successively refined during the testing process (Samples 1, 2 and 3). The results are shown in FIG. 3, in which the thickness measurements are compared statistically by probability theory using a normal distribution.

Sample 1, an early prototype, has a standard deviation of 0.0352, which is greater than the standard deviation 0.0241 of the control, indicating a lower uniformity of thickness. Samples 2 and 3, printed after G-code optimisation, have standard deviations of 0.0258 and 0.0246 respectively, which are similar to the control. The differences in thickness uniformity of the 3D printed gloves (Samples 2 and 3) are therefore similar to that of a conventional dipped glove. However, in the conventional dipped glove the thickness increases from the cuff region to the fingertip region, resulting in poor sensitivity, whereas in the 3D printed gloves the variations in thickness are distributed randomly, resulting in generally better sensitivity in the fingertips.

With regard to average thickness, the 3D printed gloves are thinner with mean values of only 0.093 mm for Samples 2 and 3, whereas the conventional control glove has a mean thickness of 0.2122 mm. The thickness of the 3D printed gloves can be controlled by adjusting the number of layers of latex applied during the printing process.

### Mechanical Testing

Tensile testing results for maximum stress and maximum strain are shown in FIGS. 6 & 7. The 3D printed gloves showed larger maximum strength but were still in a similar range to the conventional control glove. The mean maximum strength of the conventional control glove was 10.72 MPa while the 3D printed gloves had means of 15.30 MPa and 15.02 MPa for Samples 1 and 2 respectively.

Gloves incorporating 0.15% wt graphene platelets have also been successfully manufactured using the 3D printing process and are currently being tested.

### Manufacturing Analysis

A simplified analysis for assessing productivity has been made, assuming that the novel 3D printed technology is fully developed and adjusted for industrial use. These assumptions are considered to be feasible with future research by correctly adjusting the parameters of the process and creating a customised compounding of the material.

TABLE 1
General information.

| | |
|---|---|
| Density (Average) [g/ml] | 0.95646 |
| Mass of glove (size 7.5) [g] | 12.31 |
| Nozzle specifications | ⅛, SAM-01-02, 0.2 bar |
| | liquid, 2.5 bar air |
| Nominal Flow [l/h] | 2.7 |
| Solution | 60% solid particles |
| | Latex, diluted by 20% |
| Material Efficiency [%] | 34 |
| Solid Fraction [%] | 41.7 |
| Solid Latex Spray Flow [g/h] | 885.45 |
| Hour/year | 8760 |
| Equipment Availability | 90% |
| Glove surface Area size 7.5 [mm2] | 506 |
| Spray width [mm] | 10 |

### Process

The analysis considers the minimum time needed to deposit the whole glove material using the mass of the glove divided by the mass flow of the spray. Table 2 shows the estimated glove production by hourly rate using different number of nozzles and three different material efficiencies.

TABLE 2
Estimated Glove Production per Hour by
number of nozzles and by material efficiency.
Production [Glove/Hour]

| | Material Efficiency [%] | | |
|---|---|---|---|
| Nozzles | 60% | 80% | 100% |
| 1 | 52 | 70 | 87 |
| 2 | 104 | 139 | 174 |
| 3 | 157 | 209 | 261 |
| 4 | 209 | 278 | 348 |

### Layers

The number of layers is relevant to be able to customise the properties of the gloves with the use of different materials in each layer. This number of layers was calculated using different speeds of a CNC linear axis and comparing with the spraying time that was calculated using the flow of the spraying nozzle. The results (FIG. 8) show the potential layers at different speeds of the CNC print head.

1. A method of manufacturing a latex rubber article, the method comprising:
a. providing a former wherein at least a part of the former comprises a mould surface that forms the shape of the latex rubber article;
b. applying liquid latex to the mould surface using an applicator, wherein the applicator is configured to apply the liquid latex to an applicator area that is smaller than the mould surface;
c. providing relative movement between the applicator and the former to produce a latex coating that covers the mould surface;
d. curing the latex coating on the former to form the latex rubber article; and
e. removing the latex rubber article from the former.

2. A method according to claim 1, wherein the relative movement between the applicator and the former is automatically controlled via a controller.

3. A method according to claim 1, wherein the liquid latex comprises an aqueous dispersion that includes polymer particles in an amount ranging between about 40% wt. to about 70% wt., or about 45% wt. to about 65% wt., or about 50% wt. to 60% wt., relative to the total % wt. of the aqueous dispersion.

4. A method according to claim 1, wherein the liquid latex comprises ammonia in an amount ranging from about 0.2% wt. to about 10% wt., or about 0.2% wt. to about 2% wt., or about 0.2% wt. to about 1% wt., relative to the total % wt. of the liquid latex solution.

5. A method according to claim 1, wherein the liquid latex comprises an additive selected from the group comprising ceramic powders, carbon materials, nanomaterials, 2D materials, boron nitride, graphene, 1D materials, carbon nanotubes, bismuth oxide, iron oxide, ferrite and carbon.

6. A method according to claim 1, wherein the applicator area is less than 20 cm², or less than 10 cm², or less than 5 cm².

7. A method according to claim 1, further comprising applying the liquid latex to a plurality of application areas, the plurality of application areas comprising at least a first application area and a second application area.

8. A method according to claim 7, further comprising applying the liquid latex to the plurality of application areas simultaneously.

9. A method according to claim 7, further comprising applying the liquid latex to the first application area and the second application area to provide the first application area with a first portion of the latex coating and the second application area with a second portion of the latex coating.

10. A method according to claim 9, wherein the first portion and the second portion comprise different thicknesses.

11. A method according to claim 9, wherein the first portion and the second portion comprise different liquid latex compositions.

12. A method according to claim 7, further comprising overlapping the first application area at least partially with the second application area.

13. A method according to claim 1, further comprising applying the liquid latex to the mould surface to provide a coating thickness of between 80 μm and 500 μm, or between 100 μm and 500 μm, or between 150 μm and 250 μm.

14. A method according to claim 1, further comprising applying the liquid latex to the mould surface in a plurality of layers to produce the latex coating, the plurality of layers comprising at least a first layer and a second layer.

15. A method according to claim 14, wherein each layer has a thickness of between 10 μm and 200 μm, or between 20 μm and 150 μm, or between 40 μm and 100 μm.

16. A method according to claim 15, further comprising applying the liquid latex to the mould surface to form the first layer, and applying the liquid latex to the first layer to form the second layer, such that layer-by-layer deposition is used to produce the latex coating.

17. A method according to claim 15, further comprising applying the liquid latex to the first layer to form the second layer after the first layer has at least partially cured.

18. A method according to claim 15, further comprising applying a first liquid latex to form the first layer and applying a second liquid latex to form the second layer, the first liquid latex and the second liquid latex comprising different liquid latex compositions.

19. A method according to claim 15, wherein the first layer and the second layer comprise different thicknesses.

20. A method according to claim 1, wherein the former comprises a ceramic material.

21. A method according to claim 1, wherein the former comprises a hand-shaped mould surface and the latex rubber article comprises a latex glove.

22. A method according to claim 1, wherein the applicator comprises one or more spraying nozzles.

23. A method according to claim 1, further comprising adjusting the applicator relative to the former to alter the angle of application of the liquid latex to the mould surface.

24. A method according to claim 1, further comprising providing a plurality of applicators, wherein each applicator is independently adjustable.

25. A method according to claim 1, further comprising heating the former to cure the liquid latex applied to the mould surface and form a layer of cured latex on the mould surface.

26. A method according to claim 25, further comprising applying additional liquid latex to the layer of cured latex on the mould surface.

27. A method according to claim 25, further comprising heating the former using an internal heater, optionally an internal resistance heater.

28. A method according to claim 25, further comprising heating the former using an external heater.

29. A method according to claim 25, further comprising heating the former to a temperature in the range between 20° C. and 160° C., or between 20° C. and 100° C., or between 20° C. and 60° C.

30. A method according to claim 25, wherein heating the former comprises heating the former before and/or while applying liquid latex to the mould surface.

31. A method according to claim 25, further comprising heating the former to a first temperature while applying liquid latex to the former and heating the former to a second temperature after applying liquid latex to the former, wherein the second temperature is higher than the first temperature.

32. A method according to claim 31, wherein the first temperature comprises heating the former using an internal heater or an external heater, and the second temperature comprises heating the former using the internal heater and an external heater.

33. A method according to claim 1, wherein providing relative movement between the applicator and the former comprises providing relative rotation about an axis (X).

34. A method according to claim 33, wherein providing relative movement between the former and the applicator further comprises providing relative movement in a direction that is substantially parallel or perpendicular to the axis (X).

35. A latex rubber article manufactured by a method as defined by claim 1.

36. A latex rubber article as claimed in claim 35, wherein the latex rubber article is a latex rubber glove that comprises a plurality of glove portions, including a palmar portion, a dorsal portion and a finger portion.

37. A latex rubber article as claimed in claim 36, wherein at least one of said glove portions has a uniform thickness distribution with a standard deviation of less than 0.035, or less than 0.03, or less than 0.025.

38. A latex rubber article as claimed in claim 36, wherein the latex rubber glove comprises at least a first region and a second region, the first region and the second region comprising different thicknesses.

39. A latex rubber article as claimed in claim 38, wherein the first region comprises the finger portion of the latex rubber glove, and the thickness is less in the first region than the second region.

40. A latex rubber article as claimed in claim 38, wherein the second region comprises a cuff region of the latex rubber glove, and the thickness is greater in the second region than the first region.

41. A latex rubber article as claimed in claim 36, wherein the latex rubber in at least one of the glove portions comprises an additive from a group comprising ceramic powders, carbon materials, nanomaterials, 2D materials, boron nitride, graphene, 1D materials, carbon nanotubes, bismuth oxide, iron oxide, ferrite and carbon.

42. An apparatus for manufacturing the latex rubber article according to the method of claim 1 comprising:
a former wherein at least a part of the former comprises a mould surface that forms the shape of the latex rubber article;
an applicator configured to apply liquid latex to an applicator area that is smaller than the mould surface;
a drive means configured to provide relative movement between the applicator and the former to produce a latex coating that covers the mould surface; and
a heater for heating the former configured to cure the latex coating on the former to form the latex rubber article or a first layer of the latex rubber article.

43. The apparatus according to claim 42, further comprising a chamber configured to house at least a part of the former and/or the applicator and/or the drive means and/or the heater.

44. The apparatus according to claim 42, wherein the heater comprises an internal heater, optionally an internal resistance heater.

45. The apparatus according to claim 42 to 44, wherein the heater comprises an external heater.

46. The apparatus according to claim 42, wherein the drive means comprises a controller configured to automatically control the relative movement between the applicator and the former.

47. The apparatus according to claim 42, further comprising a removal means configured to remove the latex rubber article from the former.

48. The apparatus according to claim 47, wherein the removal means comprises a pressured air supply configured to force the latex rubber article off the former.
